(19) United States
(12) Patent Application Publication
(10) Pub. No.: US 2005/0283051 A1
(43) Pub. Date: Dec. 22, 2005

(54) Portable health information displaying and managing device

(76) Inventor: Yu-Yu Chen, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/869,952

(22) Filed: Jun. 18, 2004

(51) Int. Cl. A63B 71/00; A61B 5/00; A61B 10/00

(52) U.S. Cl. 600/300; 482/8; 128/920

(57) ABSTRACT

A portable health information displaying and managing device includes an exercise signal detector for generating an exercise signal; a microprocessor for calculating, storing and displaying data of personal exercise and diet, calories taken/consumed, body fat value, weight, etc.; a standard body information memory for storing at least one group of standard body information values; a desired ideal body information distribution curve memory for storing at least one group of ideal body information values established or preset by a user; a current body information memory for storing at least one group of user's current body information values; a body information display for displaying the standard, the ideal, and the current body information values. The device also includes a receiver for receiving or downloading a user's exercise data from an exerciser and a transmitter for uploading the user's body information to a signal processing apparatus for further statistical analysis of such body information. A plurality of health condition lights are provided to show different colors corresponding to different conditions of the user's body information to assist in managing user's health information.

### BACKGROUND OF THE INVENTION

1. Field of the Invention

The present invention relates to a health managing device, and more particularly to a portable health information displaying and managing device.

2. Description of the Prior Art

With the prosperous developments in the modern society, people's diets are also diversified. However, improper and surplus diets and lacking in exercise are always important factors resulting in fatness and illness. In assessing a person's health condition, the weight is usually an important basis for such assessment.

Various types of exercisers and bodybuilding apparatus have been developed and introduced into the market for people living and working in the nowadays busy industrial society to conveniently do some exercises. Meanwhile, various kinds of body signal sensors or exercise signal sensors have been developed to assist people in accurately controlling their exercises and knowing their physical condition during doing exercises.

Currently, a most common way for most people to control their weight is to periodically measure the weight using a weight scale. A conventional weight scale has only one signal function of measuring the weight, and is not portable. Thus, the conventional weight scale does not satisfy general exercisers.

### SUMMARY OF THE INVENTION

A primary object of the present invention is to provide a portable health information displaying and managing device that can be carried along with a user for easy management of the user's weight.

Another object of the present invention is to provide a portable health information displaying and managing device capable of displaying weight-managing graphics. A user may set his or her target body information, and a body information display on the device of the present invention can display a comparative graphic showing a user current body information value distribution curve and an ideal body information value distribution curve, as well as a difference in percentage between the values shown in the two curves.

A further object of the present invention is to provide a portable health information displaying and managing device capable of displaying a user's weekly, monthly, or annual weight distribution curve for the user to accurately control a real condition of his body information.

A still further object of the present invention is to provide a portable health information displaying and managing device capable of providing a quick-warning function. For this purpose, the device includes a plurality of health condition lights. When a user's body information is higher than, lower than, or within a standard value or a preset value, one of the health condition lights corresponding to the displayed condition will light to show a specific color, so as to assist the user in the body information management.

To achieve the above objects, in accordance with the present invention, there is provided a portable health information displaying and managing device. The device includes an exercise signal detector for generating an exercise signal; a microprocessor for calculating, storing and displaying data of personal exercise and diet, calories taken/consumed, body fat, weight, etc.; a standard body information memory for storing at least one group of standard body information values; a desired ideal body information distribution curve memory for storing at least one group of ideal body information values established or preset by a user; a current body information memory for storing at least one group of user's current body information values; a body information display for displaying the standard, the ideal, and the current body information values. The device also includes a receiver for receiving or downloading a user's exercise data from an exerciser and a transmitter for uploading the user's body information to a signal processing apparatus for further statistical analysis of such body information. A plurality of health condition lights are provided to show different colors corresponding to different conditions of the user's body information to assist in managing user's health information.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a schematic view of a portable health information displaying and managing device according to the present invention;

FIG. 2 is a functional block diagram of the present invention;

FIG. 3 is a perspective view showing that the present invention is receiving exercise data from an exerciser on which a user is doing exercise;

FIG. 4 is a perspective view showing the present invention is receiving user's weight and body fat value from a weight and body fat scale;

FIG. 5 shows that the present invention is adapted to measure the accumulated number of steps of or the calories consumed by a user in walking or running, and show such datum on a body information display of the present invention;

FIG. 6 shows that the body information display of the present invention displays a user's current weight and compares the same with an ideal weight value, wherein a green one of three health condition lights is on to indicate the user's current weight is within the range of ideal weight;

FIG. 7 shows that the body information display of the present invention displays a user's current weight and a red one of three health condition lights is on to indicate the user's current weight is higher than the ideal weight;

FIG. 8 shows that the body information display of the present invention displays a user's current weight and a yellow one of three health condition lights is on to indicate the user's current weight is lower than the ideal weight;

FIG. 9 shows that the body information display of the present invention displays a comparative graphic showing a user past/current weight distribution curve and a desired ideal weight distribution curve, and displays above the comparative graphic a difference in percentage between the weights indicated by the two curves, wherein the red health condition light is on to indicate the user's current weight is higher than the ideal weight distribution curve;

FIG. 10 shows that the body information display of the present invention displays a comparative graphic showing a user past/current weight distribution curve and a desired ideal weight distribution curve, and displays above the comparative graphic a difference in percentage between the weights indicated by the two curves, wherein the yellow health condition light is on to indicate the user's current weight is lower than the ideal weight distribution curve;

FIG. 11 shows that the body information display of the present invention displays a comparative graphic showing a user past/current weight distribution curve and a desired ideal weight distribution curve, and displays above the comparative graphic a difference in percentage between the weights indicated by the two curves, wherein the green health condition light is on to indicate the user's current weight is within the range of the ideal weight distribution curve;

FIG. 12 shows that the body information display of the present invention displays a user's current caloric expenditure and compares the same with an ideal caloric expenditure value, wherein the green health condition light is on to indicate the user's current caloric expenditure is within the range of ideal caloric expenditure;

FIG. 13 shows that the body information display of the present invention displays a user's current caloric expenditure and the red health condition light is on to indicate the user's current caloric expenditure is higher than the ideal caloric expenditure;

FIG. 14 shows that the body information display of the present invention displays a user's current caloric expenditure and the yellow health condition light is on to indicate the user's current caloric expenditure is lower than the ideal caloric expenditure;

FIG. 15 shows that the body information display of the present invention displays a comparative graphic showing a user past/current caloric expenditure distribution curve and a desired ideal caloric expenditure distribution curve, and displays above the comparative graphic a difference in percentage between the caloric expenditures indicated by the two curves, wherein the red health condition light is on to indicate the user's current caloric expenditure is higher than the ideal caloric expenditure distribution curve;

FIG. 16 shows that the body information display of the present invention displays a comparative graphic showing a user past/current caloric expenditure distribution curve and a desired ideal caloric expenditure distribution curve, and displays above the comparative graphic a difference in percentage between the caloric expenditures indicated by the two curves, wherein the yellow health condition light is on to indicate the user's current caloric expenditure is lower than the ideal caloric expenditure distribution curve;

FIG. 17 shows that the body information display of the present invention displays a comparative graphic showing a user past/current caloric expenditure distribution curve and a desired ideal caloric expenditure distribution curve, and displays above the comparative graphic a difference in percentage between the caloric expenditures indicated by the two curves, wherein the green health condition light is on to indicate the user's current caloric expenditure is within the range of the ideal caloric expenditure distribution curve;

FIG. 18 shows that the body information display of the present invention displays a user's current body fat value and compares the same with an ideal body fat value, wherein the red health condition light is on to indicate the user's current body fat value is higher than the ideal body fat value;

FIG. 19 shows that the body information display of the present invention displays a user's current body fat value and the yellow health condition light is on to indicate the user's current body fat value is lower than the ideal body fat value;

FIG. 20 shows that the body information display of the present invention displays a user's current body fat value and the green health condition light is on to indicate the user's current body fat value is within the range of ideal body fat

FIG. 21 shows that the body information display of the present invention displays a comparative graphic showing a user past/current body fat value distribution curve and a desired ideal body fat value distribution curve, and displays above the comparative graphic a difference in percentage between the body fat values indicated by the two curves, wherein the red health condition light is on to indicate the user's current body fat value is higher the ideal body fat value distribution curve;

FIG. 22 shows that the body information display of the present invention displays a comparative graphic showing a user past/current body fat value distribution curve and a desired ideal body fat value distribution curve, and displays above the comparative graphic a difference in percentage between the body fat value indicated by the two curves, wherein the yellow health condition light is on to indicate the user's current body fat value is lower the ideal body fat value distribution curve; and

FIG. 23 shows that the body information display of the present invention displays a comparative graphic showing a user past/current body fat value distribution curve and a desired ideal body fat value distribution curve, and displays above the comparative graphic a difference in percentage between the body fat value indicated by the two curves, wherein the green health condition light is on to indicate the user's current body fat value is within the range of ideal body fat value distribution curve.

### DETAILED DESCRIPTION OF THE PREFERRED EMBODIMENTS

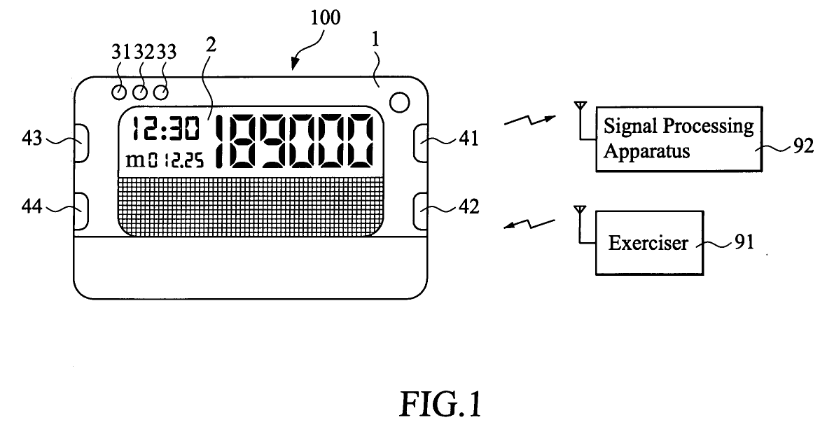

Please refer to FIG. 1 that is a schematic view of a portable health information displaying and managing device 100 according to the present invention. As shown, the portable health information displaying and managing device 100 mainly includes a case 1, on which a body information display 2 and a plurality of health condition lights 31 (red), 32 (yellow), and 33 (green) are provided. Moreover, the case 1 is also provided at predetermined positions with a plurality of setting keys 41, 42, 43, and 44. The setting keys 41, 42, 43, and 44 are provided for the user to input his personal body information including weight, body fat value and so on or to set his desired ideal body information values.

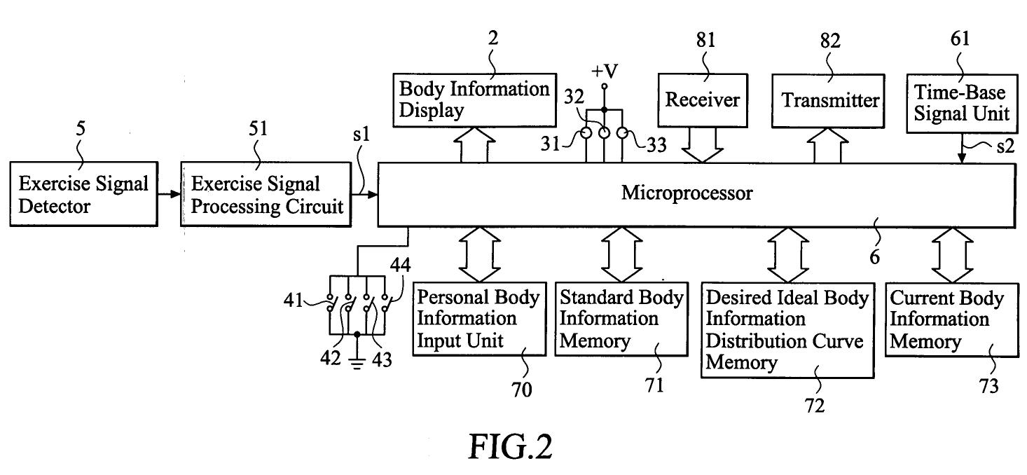

FIG. 2 is a functional circuit block diagram of the portable health information displaying and managing device 100 of the present invention. As shown, the present invention includes an exercise signal detector 5, which may be, for example, a conventional acceleration sensor, and is adapted to detect the number of steps and the speed of an exerciser in jogging or running.

A signal detected by the exercise signal detector 5 is sent to and processed at an exercise signal processing circuit 51 to generate an exercise signal s1, which is sent to a microprocessor 6. The exercise signal processing circuit 51 may include a conventional amplifier, a filtering circuit, and a signal shaping circuit.

The microprocessor 6 is capable of calculating, storing, and displaying data about personal exercise and diet, calories taken/consumed, body fat value, weight, etc.

The microprocessor 6 is electrically connected to the body information display 2 to allow display of a user's body information on the body information display 2. The microprocessor 6 is also electrically connected to the health condition lights 31, 32, and 33. When the user's body information is higher than, lower than, or within a range of standard value or preset value, the corresponding health condition light 31, 32, or 33 will light to show a different color as a warning to the user, so as to achieve the object of body information management. In the illustrated embodiment of the present invention, a red light 31 lights when the user's body information is higher than a target value, a green light 33 lights when the user's body information is within the range of target value, and a yellow light 32 lights when the user's body information is lower than the target value.

The microprocessor 6 is further electrically connected to a standard body information memory 71, a desired ideal body information distribution curve memory 72, and a current body information memory 73. In the standard body information memory 71, there are stored values of standard weight, calories taken/consumed, and/or body fat. For example, the standard weight values include male standard weights in kilogram calculated using the formula of: (height in centimeter−80)×0.7 or 62+(height in centimeter−170)×0.6; and female standard weights in kilogram calculated using the formula of (height in centimeter−70)×0.6 or 52+(height in centimeter−158)×0.5. The weight value can be expressed in other conventional units e.g. pound. In the desired ideal body information distribution curve memory 72, there are stored ideal weight values established or preset by the user. And, in the current body information memory 73, there is stored the user's current body information.

Please refer to FIGS. 1 and 3. The microprocessor 6 is also electrically connected to a receiver 81 for receiving or downloading a user's exercise data from an exerciser 91 on which the user are doing exercises, and a transmitter 82 for uploading the user's body information to a signal processing apparatus 92, such as, for example, a personal digital assistant (PDA), a portable computer, or a personal computer, so as to conduct further statistical analysis of such body information.

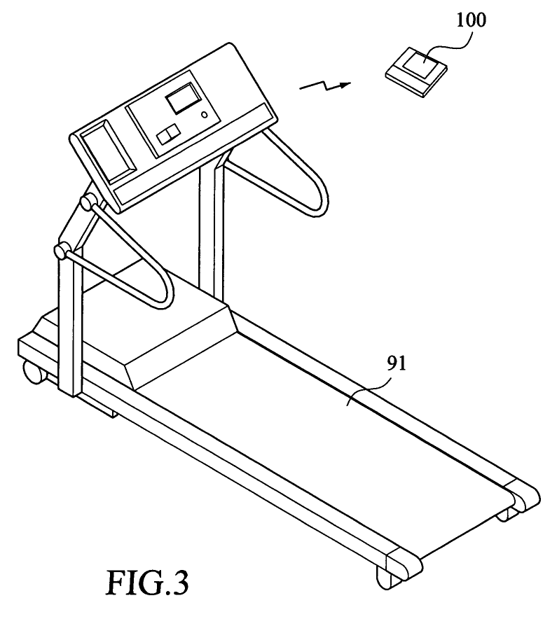

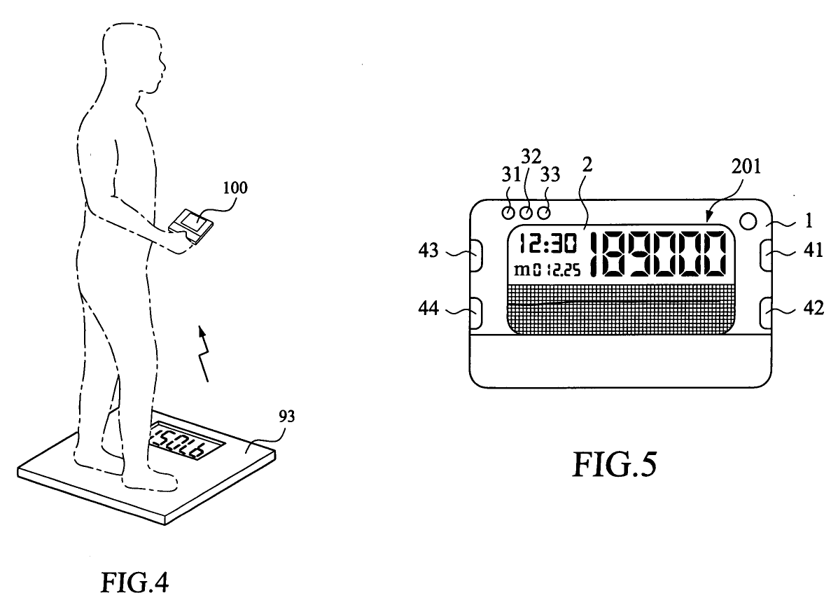

A user may conveniently hold the portable health information displaying and managing device 100 of the present invention with a hand, as shown in FIG. 4, or put it in a pocket. When the user stands on a weight and body fat scale 93, the portable health information displaying and managing device 100 is able to wirelessly receive values about the user's weight and body fat from the weight and body fat scale 93.

Moreover, the microprocessor 6 is electrically connected to a time-base signal unit 61, so as to receive a time-base signal s2 from the time-base signal unit 61. The microprocessor 6 is also electrically connected to a personal body information input unit 70, so that a user may input at any time his or her current body information and other personal health data to the portable health information displaying and managing device 100.

Please refer to FIG. 5 that shows the present invention is adapted to measure the number of steps of or the calories consumed by a user in walking or running, and show such information 201 on the body information display 2 of the present invention.

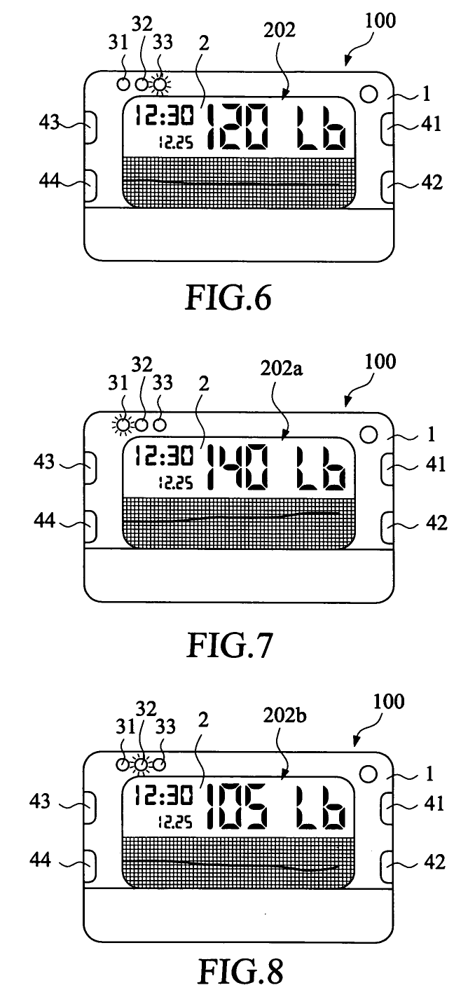

FIG. 6 shows that the body information display 2 of the present invention displays a user's current weight 202 and the green health condition light 33 is on to indicate the user's current weight is within the range of ideal weight.

FIG. 7 shows that the body information display 2 of the present invention displays a user's current weight 202a and the red health condition light 31 is on to indicate the user's current weight is higher than the ideal weight.

FIG. 8 shows that the body information display 2 of the present invention displays a user's current weight 202b and the yellow health condition light 32 is on to indicate the user's current weight is lower than the ideal weight.

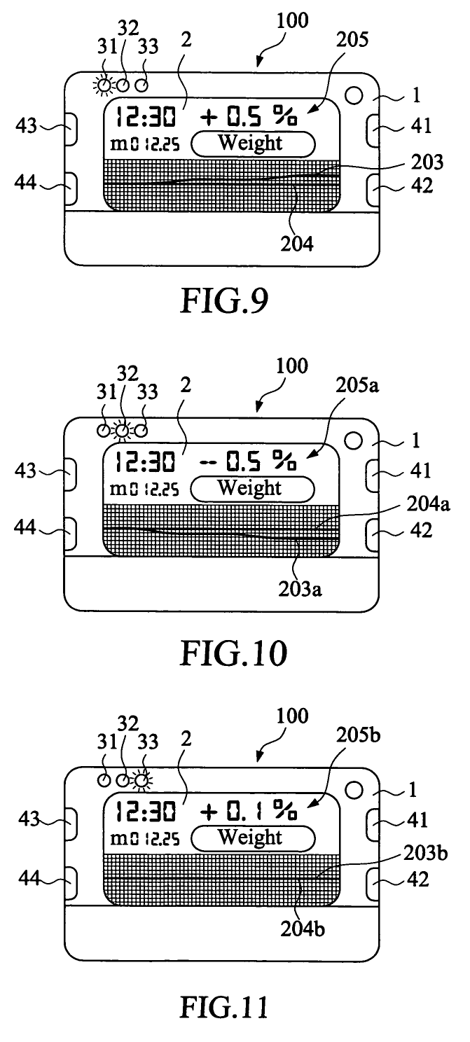

FIG. 9 shows that the body information display 2 of the present invention displays a comparative graphic showing a user past/current weight distribution curve 203 and a desired ideal weight distribution curve 204, and displays above the comparative graphic a difference in percentage 205 between the weights indicated by the two curves. Meanwhile, the red health condition light 31 is on to indicate the user's current weight is higher than the ideal weight distribution curve.

FIG. 10 shows that the body information display 2 of the present invention displays a comparative graphic showing a user past/current weight distribution curve 203a and a desired ideal weight distribution curve 204a, and displays above the comparative graphic a difference in percentage 205a between the weights indicated by the two curves. Meanwhile, the yellow health condition light 32 is on to indicate the user's current weight is lower than the ideal weight distribution curve.

FIG. 11 shows that the body information display 2 of the present invention displays a comparative graphic showing a user past/current weight distribution curve 203b and a desired ideal weight distribution curve 204b, and displays above the comparative graphic a difference in percentage 205b between the weights indicated by the two curves. Meanwhile, the green health condition light 33 is on to indicate the user's current weight is within the range of the ideal weight distribution curve.

In addition to the display of a user's weight and different weight distribution curves on the body information display 2, the present invention may also be applied to display other useful health managing information on the body information display 2. For example, it is also possible to display on the body information display 2 caloric expenditure and caloric expenditure distribution curves, or a user's body fat value and body fat value distribution curves for the user's reference.

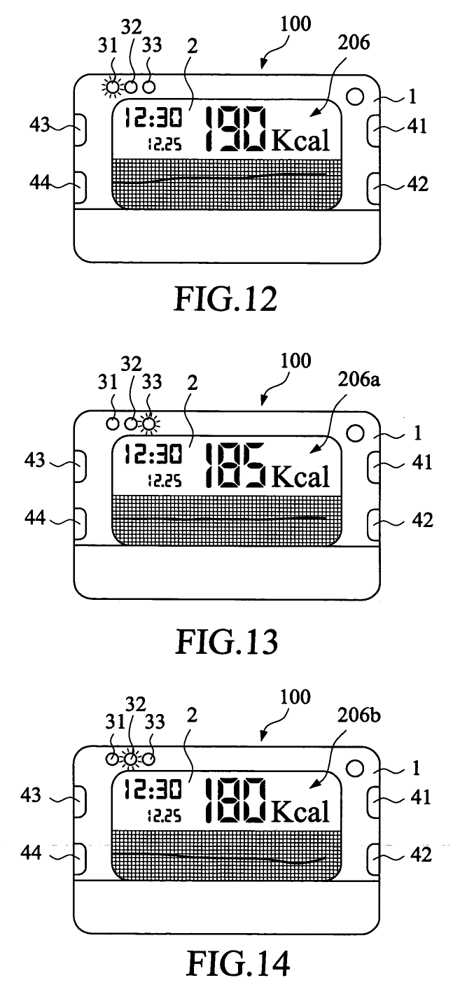

Please refer to FIG. 12. FIG. 12 shows that the body information display 2 of the present invention displays a user's current caloric expenditure 206 and compares the same with an ideal caloric expenditure value and the red health condition light 31 is on to indicate the user's current caloric expenditure is higher than the ideal caloric expenditure.

FIG. 13 shows that the body information display 2 of the present invention displays a user's current caloric expenditure 206a and the green health condition light 33 is on to indicate the user's current caloric expenditure is within the range of ideal caloric expenditure.

FIG. 14 shows that the body information display 2 of the present invention displays a user's current caloric expenditure 206b and the yellow health condition light 32 is on to indicate the user's current caloric expenditure is lower than the ideal caloric expenditure.

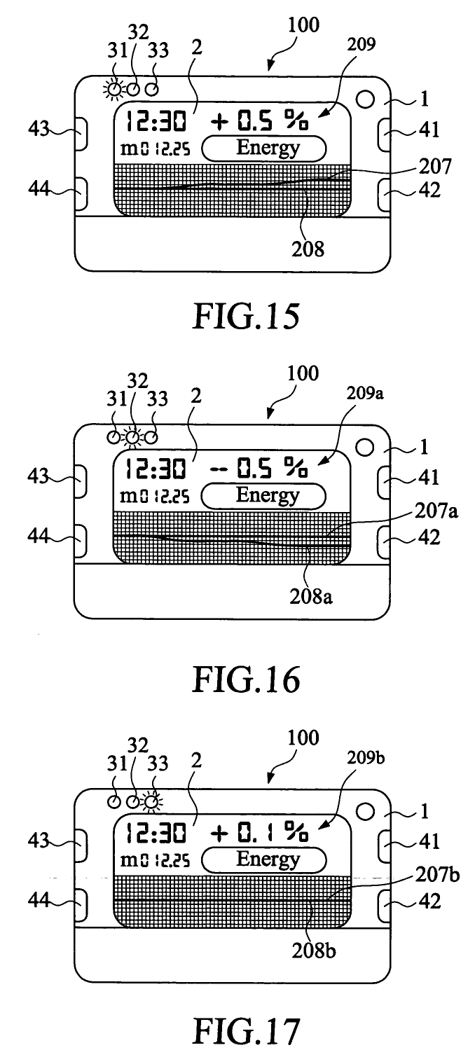

FIG. 15 shows that the body information display 2 of the present invention displays a comparative graphic showing a user past/current caloric expenditure distribution curve 207 and a desired ideal caloric expenditure distribution curve 208, and displays above the comparative graphic a difference in percentage 209 between the caloric expenditures indicated by the two curves. Meanwhile, the red health condition light 31 is on to indicate the user's current caloric expenditure is higher than the ideal caloric expenditure distribution curve.

FIG. 16 shows that the body information display 2 of the present invention displays a comparative graphic showing a user past/current caloric expenditure distribution curve 207a and a desired ideal caloric expenditure distribution curve 208a, and displays above the comparative graphic a difference in percentage 209a between the caloric expenditures indicated by the two curves. Meanwhile, the yellow health condition light 32 is on to indicate the user's current caloric expenditure is lower than the ideal caloric expenditure distribution curve.

FIG. 17 shows that the body information display 2 of the present invention displays a comparative graphic showing a user past/current caloric expenditure distribution curve 207b and a desired ideal caloric expenditure distribution curve 208b, and displays above the comparative graphic a difference in percentage 209b between the caloric expenditures indicated by the two curves. Meanwhile, the green health condition light 33 is on to indicate the user's current caloric expenditure is within the range of the ideal caloric expenditure distribution curve.

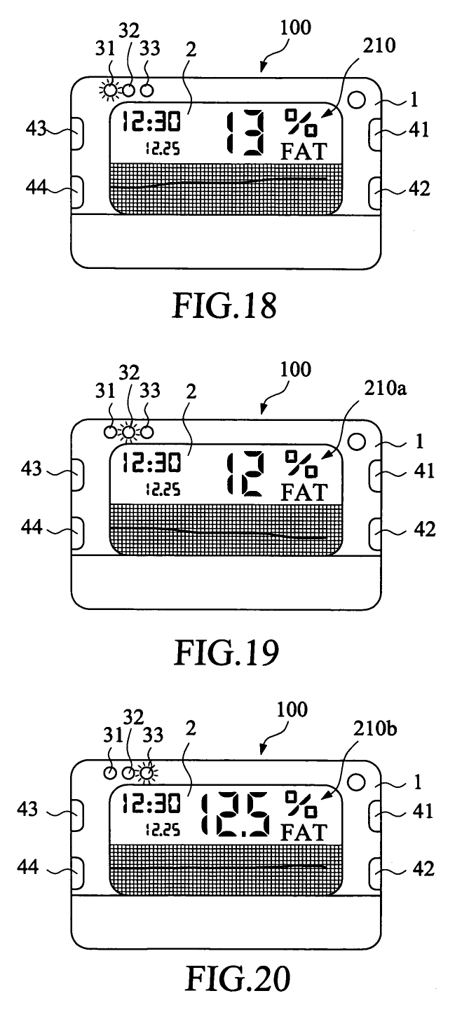

FIG. 18 shows that the body information display 2 of the present invention displays a user's current body fat value 210 and compares the same with an ideal body fat value and the red health condition light 31 is on to indicate the user's current body fat value 210 is higher than the ideal body fat value.

FIG. 19 shows that the body information display 2 of the present invention displays a user's current body fat value 210a and the yellow health condition light 32 is on to indicate the user's current body fat value 210a is lower than the ideal body fat value.

FIG. 20 shows that the body information display 2 of the present invention displays a user's current body fat value 210b and the green health condition light 33 is on to indicate the user's current body fat value 210b is within the range of ideal body fat value.

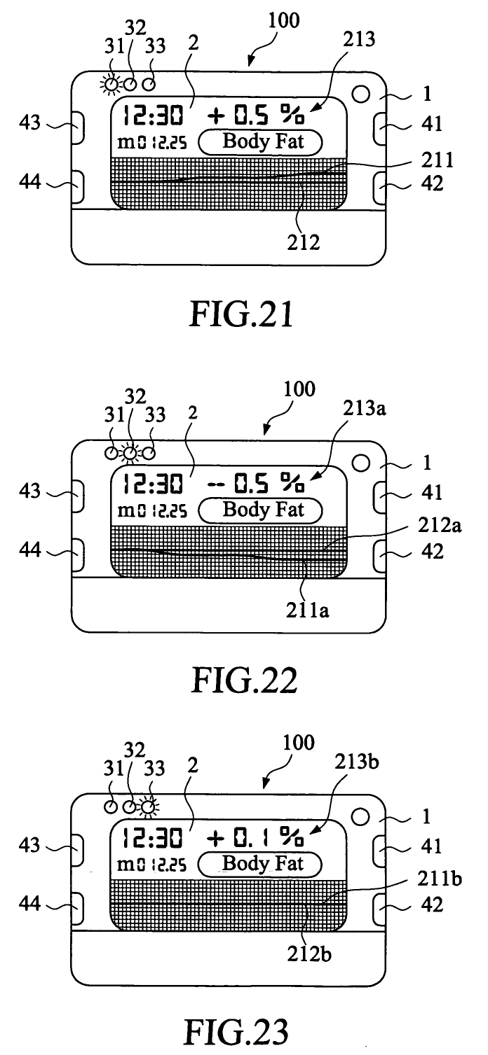

FIG. 21 shows that the body information display 2 of the present invention displays a comparative graphic showing a user past/current body fat value distribution curve 211 and a desired ideal body fat value distribution curve 212, and displays above the comparative graphic a difference in percentage 213 between the body fat values indicated by the two curves. Meanwhile, the red health condition light 31 is on to indicate the user's current body fat value is higher the ideal body fat value distribution curve.

FIG. 22 shows that the body information display 2 of the present invention displays a comparative graphic showing a user past/current body fat value distribution curve 212a and a desired ideal body fat value distribution curve 211a, and displays above the comparative graphic a difference in percentage 213a between the body fat values indicated by the two curves. Meanwhile, the yellow health condition light 32 is on to indicate the user's current body fat value is lower the ideal body fat value distribution curve.

FIG. 23 shows that the body information display 2 of the present invention displays a comparative graphic showing a user past/current body fat value distribution curve 211b and a desired ideal body fat value distribution curve 212b, and displays above the comparative graphic a difference in percentage 213b between the body fat values indicated by the two curves. Meanwhile, the green health condition light 33 is on to indicate the user's current body fat value is within the range of ideal body fat value distribution curve.

The present invention has been described with some preferred embodiments thereof and it is understood that many changes and modifications in the described embodiments can be carried out without departing from the scope and the spirit of the invention that is intended to be limited only by the appended claims.

1. A portable health information displaying and managing device, comprising:
an exercise signal detector for detecting a user's physical condition during exercising, said physical condition during exercising being sent to and processed at an exercise signal processing circuit to generate an exercise signal;
a microprocessor for inputting data, and calculating, storing and displaying data at least including the user's personal exercise information, calories taken/consumed, body fat, weight;
a standard body information memory electrically connected to said microprocessor for storing at least one group of standard body information values;
a desired ideal body information distribution curve memory electrically connected to said microprocessor for storing at least one group of ideal body information values established or preset by a user;
a current body information memory electrically connected to said microprocessor for storing at least one group of user's current body information values; and
a body information display for displaying said standard body information values, said ideal body information values, and said current body information values.

2. The portable health information displaying and managing device as claimed in claim 1, wherein said standard body information memory stores at least one group of standard weight values; said desired ideal body information distribution curve memory stores at least one group of desired ideal weight distribution curves; and said current body information memory stores at least one group of current weight values.

3. The portable health information displaying and managing device as claimed in claim 1, wherein said standard body information memory stores at least one group of standard caloric expenditures; said desired ideal body information distribution curve memory stores at least one group of desired ideal caloric expenditure distribution curves; and said current body information memory stores at least one group of current caloric expenditure.

4. The portable health information displaying and managing device as claimed in claim 1, wherein said standard body information memory stores at least one group of standard body fat values; said desired ideal body information distribution curve memory stores at least one group of desired ideal body fat value distribution curves; and said current body information memory stores at least one group of current body fat values.

5. The portable health information displaying and managing device as claimed in claim 1, further comprising:
a receiver electrically connected to said microprocessor for downloading the user's exercise data from an exerciser on which a user is doing exercises; and
a transmitter electrically connected to said microprocessor for uploading received data to a signal processing apparatus for further statistical analysis of such data.

6. The portable health information displaying and managing device as claimed in claim 5, wherein said receiver is a wireless receiver, and said transmitter is a wireless transmitter.

7. The portable health information displaying and managing device as claimed in claim 5, wherein said receiver is a wired receiver, and said transmitter is a wired transmitter.

8. The portable health information displaying and managing device as claimed in claim 1, further comprising a plurality of health condition lights electrically connected to said microprocessor, whereby when a user's body information displayed on said body information display is higher than, lower than, or within a range of standard value or preset value, one of said health condition lights corresponding to a real condition of said displayed user body information is on to show a specific color and thereby assists in managing the user's health information.

9. The portable health information displaying and managing device as claimed in claim 1, wherein said body information display further displays a comparative graphic showing a user past/current body information distribution curve and a desired ideal body information distribution curve.

10. The portable health information displaying and managing device as claimed in claim 1, wherein said body information display further displays a comparative graphic showing a user past/current body information distribution curve and a desired ideal body information distribution curve, and a difference in percentage between values shown on said two curves.

11. The portable health information displaying and managing device as claimed in claim 1, further comprising a personal body information input unit electrically connected to said microprocessor for a user to input his or her current body information and other personal health data.
